(19) United States
(12) Patent Application Publication
(10) Pub. No.: US 2021/0298648 A1
(43) Pub. Date: Sep. 30, 2021

(54) CALIBRATION OF A NONINVASIVE PHYSIOLOGICAL CHARACTERISTIC SENSOR BASED ON DATA COLLECTED FROM A CONTINUOUS ANALYTE SENSOR

(71) Applicant: MEDTRONIC MINIMED, INC., Northridge, CA (US)

(72) Inventors: Jesse Hefner, Minneapolis, MN (US); Nicholas S. Mairs, Minneapolis, MN (US); Brian Ross, Maple Grove, MN (US); Matthew N. Haggerty, Minnetonka, MN (US)

(21) Appl. No.: 16/828,269

(22) Filed: Mar. 24, 2020

(51) Int. Cl. A61B 5/1495; A61B 5/145; A61B 5/00; A61B 5/053

(52) U.S. Cl. CPC A61B 5/1495; A61B 5/14532; A61B 5/6802; A61B 2562/0219; A61B 5/0022; A61B 5/0024; A61B 5/0531

(57) ABSTRACT

A method of measuring a physiological characteristic of a user obtains first calibrating sensor data indicative of an analyte level of the user, and produced in response to operation of a continuous analyte sensor device during a calibration period, and obtains second calibrating sensor data indicative of status or condition of the user, and produced in response to operation of a noninvasive sensor device, concurrently with operation of the continuous analyte sensor device, during the calibration period. A correlation measurement is calculated by comparing the first calibrating sensor data against the second calibrating sensor data. When the correlation measurement exceeds a threshold value, a transfer function is generated to calculate estimated values of the analyte level as a function of sensor data from the noninvasive sensor device. The transfer function is provided to a client device associated with the user.

### TECHNICAL FIELD

Embodiments of the subject matter described herein relate generally to physiological characteristic sensors, activity tracker devices, health monitoring systems, and related operating methodologies. More particularly, the disclosed subject matter relates to a noninvasive physiological sensor device that can be calibrated using data collected from a continuous analyte sensor.

### BACKGROUND

A variety of noninvasive physiological characteristic sensor devices, activity tracker devices, and wellness monitoring devices have been developed. For example, an activity tracking device is a user-worn or carried device for monitoring and tracking fitness related metrics during an activity such as walking, running, swimming, cycling, etc. Fitness related metrics include activity distance, such as distance walked, activity time, speed, elevation changes, estimated calories burned during the activity, and heartbeat. Many activity monitoring devices are computers that are wearable or are able to be carried during the activity, such as with smartphones or smartwatches. Activity/Fitness monitoring devices can include a multitude of sensors including GPS receivers, motion sensors such as accelerometers and gyroscopes, altimeters, heart rate monitors including optical sensors and electrodes, thermometers, stress and hydration sensors—electrodermal activity, electrodes to measure impedance of the skin, optical sensors to measure oxygen levels, and the like. Various software platforms, accessible through a smartphone, a web browser, and other modalities are available for logging activity metrics so that a user can review a past exercise activity, such as a run, and so that the user can compare the activity metrics with the user's historical exercise activities. A noninvasive sensor can be designed to obtain an estimated glucose level of the user, but the obtained readings may not be accurate enough for diabetes diagnosis or for use in controlling blood glucose via insulin therapy.

### BRIEF SUMMARY

A method of measuring a physiological characteristic of a user is disclosed here. An exemplary embodiment of the method includes the steps of: obtaining first calibrating sensor data that is indicative of an analyte level of the user, the first calibrating sensor data produced in response to operation of a continuous analyte sensor device during a calibration period; obtaining second calibrating sensor data that is indicative of status or condition of the user, the second calibrating sensor data produced in response to operation of at least one noninvasive sensor device, concurrently with operation of the continuous analyte sensor device, during the calibration period; and calculating a correlation measurement by comparing the obtained first calibrating sensor data against the obtained second calibrating sensor data. When the correlation measurement exceeds a threshold value, the method generates a transfer function that calculates estimated values of the analyte level of the user as a function of noninvasive sensor data produced in response to operation of the at least one noninvasive sensor device. After generating the transfer function, the method obtains noninvasive sensor input data that is indicative of status or condition of the user, the noninvasive sensor input data produced in response to operation of the at least one noninvasive sensor device in a measurement mode. The method continues by applying the transfer function to calculate, from the obtained noninvasive sensor input data, an estimated value of the analyte level of the user; and communicating the estimated value of the analyte level for presentation to the user.

Also disclosed here is a non-transitory computer-readable storage medium having program instructions stored thereon, wherein the program instructions are configurable to cause at least one processor device to perform a method that involves the steps of: obtaining first calibrating sensor data that is indicative of an analyte level of the user, the first calibrating sensor data produced in response to operation of a continuous analyte sensor device during a calibration period; obtaining second calibrating sensor data that is indicative of status or condition of the user, the second calibrating sensor data produced in response to operation of at least one noninvasive sensor device, concurrently with operation of the continuous analyte sensor device, during the calibration period; calculating a correlation measurement by comparing the obtained first calibrating sensor data against the obtained second calibrating sensor data; when the correlation measurement exceeds a threshold value, generating a transfer function that calculates estimated values of the analyte level of the user as a function of noninvasive sensor data produced in response to operation of the at least one noninvasive sensor device; and providing the generated transfer function to a client device associated with the user.

A system to measure a physiological characteristic of a user is also disclosed here. An exemplary embodiment of the system includes at least one processor device and a non-transitory processor-readable medium operatively associated with the at least one processor device. The processor-readable medium stores executable program instructions configurable to cause the at least one processor device to perform a method that includes the steps of: obtaining first calibrating sensor data that is indicative of an analyte level of the user, the first calibrating sensor data produced in response to operation of a continuous analyte sensor device during a calibration period; obtaining second calibrating sensor data that is indicative of status or condition of the user, the second calibrating sensor data produced in response to operation of at least one noninvasive sensor device, concurrently with operation of the continuous analyte sensor device, during the calibration period; calculating a correlation measurement by comparing the obtained first calibrating sensor data against the obtained second calibrating sensor data; when the correlation measurement exceeds a threshold value, generating a transfer function that calculates estimated values of the analyte level of the user as a function of noninvasive sensor data produced in response to operation of the at least one noninvasive sensor device; and providing the generated transfer function to a client device associated with the user. The client device obtains noninvasive sensor input data that is indicative of status or condition of the user, the noninvasive sensor input data produced in response to operation of the at least one noninvasive sensor device in a measurement mode. The client device applies the transfer function to calculate, from the obtained noninvasive sensor input data, an estimated value of the analyte level of the user.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### BRIEF DESCRIPTION OF THE DRAWINGS

A more complete understanding of the subject matter may be derived by referring to the detailed description and claims when considered in conjunction with the following figures, wherein like reference numbers refer to similar elements throughout the figures.

FIG. 1 is a block diagram representation of an exemplary embodiment of a system for measuring a physiological characteristic of a user, as arranged during a calibration period of operation;

FIG. 2 is a block diagram representation of an exemplary embodiment of a computer-based or processor-based device suitable for deployment in the system shown in FIG. 1;

FIG. 3 is a block diagram representation of the system depicted in FIG. 1, as arranged during operation in a measurement mode;

FIG. 4 is a flow chart that illustrates an exemplary embodiment of a process for calibrating a noninvasive physiological characteristic sensor; and

FIG. 5 is a flow chart that illustrates an exemplary embodiment of a process for measuring a physiological characteristic of a user.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Any implementation described herein as exemplary is not necessarily to be construed as preferred or advantageous over other implementations. Furthermore, there is no intention to be bound by any expressed or implied theory presented in the preceding technical field, background, brief summary or the following detailed description.

Techniques and technologies may be described herein in terms of functional and/or logical block components, and with reference to symbolic representations of operations, processing tasks, and functions that may be performed by various computing components or devices. Such operations, tasks, and functions are sometimes referred to as being computer-executed, computerized, software-implemented, or computer-implemented. It should be appreciated that the various block components shown in the figures may be realized by any number of hardware, software, and/or firmware components configured to perform the specified functions. For example, an embodiment of a system or a component may employ various integrated circuit components, e.g., memory elements, digital signal processing elements, logic elements, look-up tables, or the like, which may carry out a variety of functions under the control of one or more microprocessors or other control devices.

When implemented in software or firmware, various elements of the systems described herein are essentially the code segments or instructions that perform the various tasks. In certain embodiments, the program or code segments are stored in a tangible processor-readable medium, which may include any medium that can store or transfer information. Examples of a non-transitory and processor-readable medium include an electronic circuit, a semiconductor memory device, a ROM, a flash memory, an erasable ROM (EROM), a floppy diskette, a CD-ROM, an optical disk, a hard disk, or the like.

Physiological analyte sensors may be used in a variety of specialized applications. In this regard, thin film electrochemical sensors are used to test analyte levels in patients. More specifically, thin film sensors have been designed for use in obtaining a continuous, accurate, and reliable indication of blood glucose (BG) levels and for monitoring BG levels in a diabetic patient, with the distal segment portion of the sensor positioned subcutaneously in direct contact with patient extracellular fluid. Such readings can be especially useful in adjusting a treatment regimen which typically includes regular administration of insulin to the patient. Although minimally invasive continuous glucose sensors of this type generate accurate glucose levels, they can be inconvenient, expensive, and unnecessary for many users (e.g., non-diabetic users). Accordingly, it is desirable to provide an improved noninvasive physiological characteristic sensor having improved output accuracy. Furthermore, other desirable features and characteristics will become apparent from the subsequent detailed description and the appended claims, taken in conjunction with the accompanying drawings and the foregoing technical field and background.

The subject matter described here relates to noninvasive sensor devices that are capable of measuring one or more physiological characteristics of a user. More specifically, a noninvasive sensor device of the type disclosed here can be calibrated using sensor data obtained from a continuous analyte sensor device (such as a continuous glucose monitor device), and thereafter operated to produce estimated values of the analyte based on the information obtained from the noninvasive sensor(s). The non-limiting exemplary embodiment described below employs a continuous glucose sensor of the type used by diabetic patients. It should be appreciated that the concepts, operating methodologies, and calibration techniques mentioned here need not be limited to use with glucose sensors and, indeed, the concepts and technology described with reference to a glucose sensor could also be used with other medical devices, other physiological characteristic sensor types, implanted sensors or medical devices, other medical components or supplies, and the like.

For the sake of brevity, conventional aspects and technology related to glucose sensors may not be described in detail here. A glucose sensor of the type described here may be realized as an electrochemical sensor that employs the glucose oxidase enzyme. Sensors that use glucose oxidase to effect a reaction of glucose and oxygen are known, and such glucose sensors will not be described in detail here. In this regard, known and/or conventional aspects of glucose sensors and their manufacturing may be of the type described in, but not limited to: U.S. Pat. Nos. 5,391,250, 6,892,085, 7,468,033, and 7,602,310, and United States patent application numbers 2009/0299301 and 2017/0290535 (which are incorporated by reference herein).

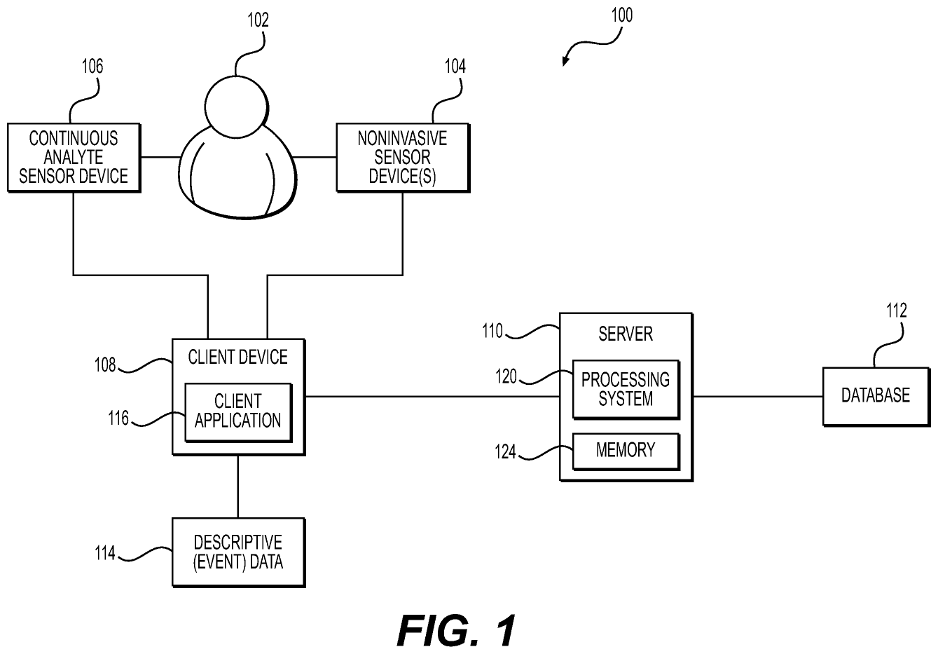

FIG. 1 depicts an exemplary embodiment of a system 100 for measuring a physiological characteristic of a user 102, as arranged during a calibration period of operation. The illustrated embodiment of the system 100 includes, without limitation: at least one noninvasive sensor device 104; a continuous analyte sensor device 106; a client device 108 that is owned by, operated by, or otherwise associated with the user 102; and a remote server system 110. The system 100 may also include or cooperate with a database system 112 associated with the server system 110. The system 100 may also include or cooperate with one or more sources of descriptive data 114, which can provide descriptive data about events, status of the user 102, operating conditions, or the like.

At least some of the components of the system 100 are communicatively coupled with one another to support data communication as needed. For this particular example, the client device 108 can communicate with the noninvasive sensor device(s) 104, the continuous analyte sensor device 106, and the server system 110 via suitable data communication links and communication protocols. In some implementations, the noninvasive sensor device(s) 104 can wirelessly communicate with the continuous analyte sensor device 106 via suitable data communication links and communication protocols. Other configurations and topologies are also contemplated here, such as a system that includes additional intermediary, interface, or data repeating devices in the data path between a sending device and a receiving device.

FIG. 1 depicts network communication links in a simplified manner. In practice, the system 100 may cooperate with and leverage any number of wireless and any number of wired data communication networks maintained or operated by various entities and providers. Accordingly, communication between the various components of the system 100 may involve multiple network links and different data communication protocols. In this regard, the network can include or cooperate with any of the following, without limitation: a local area network; a wide area network; the Internet; a personal area network; a near-field data communication link; a cellular communication network; a satellite communication network; a video services or television broadcasting network; a network onboard a vehicle; or the like. The components of the system 100 may be suitably configured to support a variety of wireless and wired data communication protocols, technologies, and techniques as needed for compatibility with the network.

Although any number of physically distinct noninvasive sensor devices 104 can be utilized with an embodiment of the system 100, this description refers to only one noninvasive sensor device 104 for simplicity. When deployed and operational, the noninvasive sensor device 104 uses appropriate sensing, detecting, monitoring, and/or interrogating elements and methodologies to noninvasively measure one or more conditions, physiological characteristics, measurable quantities, analyte levels, or status of the user 102. In certain embodiments, the noninvasive sensor device 104 is realized as: an activity tracker device, e.g., a pedometer; a physiological property monitoring device, e.g., an analyte sensor; a wellness monitoring device, e.g., a heartrate, blood pressure, or respiration monitor; or similar type of health monitoring device, e.g., a sleep monitor, that is associated with (e.g., worn by, carried by, physically linked to, or in proximity to) the user 102 for tracking movement of the user 102 during activity. For example, sonar or electromagnetic waves can be used to detect movements off the body. The noninvasive sensor device 104 is configured to output activity data providing an activity metric associated with the user's movement during the activity or exercise. Exemplary metrics include distance moved, speed, heart rate, step count, cadence, elevation change, activity time, elevation change rate, power, energy output, etc. Additional metrics or physiological characteristics obtained by the noninvasive sensor device 104 may include, without limitation: body temperature, perspiration level, glucose level, blood pressure, GPS location, heart rate (using electrodes such as ECG, optical light (PPG), force sensors, audio signals, capacitive sensors, or pressure transducer), movement (using force sensor, accelerometer, capacitive sensor, sonar, or RF waves), gesture recognition using an accelerometer, stress and hydration—electrodermal activity by measuring impedance of skin, SpO2 oxygen levels using optical techniques, lactate/VO2 Max using optical sensors, hydration/PH level using microfluidics, body composition using electrodes or optical sensors, EEG brain waves using EEG electrodes, metabolism and RQ-CO2 measurement with electrodes using bioimpedance, ketones using breath/gas measurement, cortisol using microfluidics.

The noninvasive sensor device 104 includes one or more sensing or detecting elements. Certain embodiments of the noninvasive sensor device 104 also include a display device, data storage, a processor, and a wireless interface (which can be used to establish and maintain a wireless communication link with the client device 108). The sensing or detecting elements may include one or more of the following, without limitation: colored light emitting diodes (LEDs); a GPS receiver; a motion sensor such as an accelerometer or gyroscope; an altimeter; a thermometer; and a heart rate monitor. Additional or alternative activity sensors can be included if so desired. A GPS receiver includes antennas that use a satellite-based navigation system with a network of satellites in orbit around the earth to provide position data. From position data, movement of a user during an activity can be accurately tracked to measure distance moved during the activity. Moreover, speed and time data for the activity is derivable from the GPS position data. An altimeter measures atmospheric pressure and derives height above sea level (or some other reference plane) based thereon. In alternative embodiments, elevation data is derived from the GPS position data or elevation data is derived from a combination of the GPS position data and measurements from the altimeter. Motion sensors such as multi-axis accelerometers and gyroscopes allow the noninvasive sensor device 104 to differentiate types of activities and also to count steps during walking and running (cadence data). One exemplary type of heart rate sensor is an optical heart rate sensor configured to direct light against the skin and to detect changes of reflectivity with heart beats. ECG type heart rate sensors are another possibility for measuring heart rate.

The continuous analyte sensor device 106 is designed, configured, arranged, and deployed to measure an analyte level of the user 102 using appropriate sensing technology. The continuous analyte sensor device 106 generates raw sensor signals or raw sensor data that represents or is otherwise indicative of the analyte level of interest. The raw information generated by the continuous analyte sensor device 106 can be converted, calibrated, translated, conditioned, or otherwise processed as needed into any desired format or usable value. For example, a raw sensor signal may be a measured current, voltage, impedance, or capacitance, which is converted into a usable or understandable value of the monitored analyte (e.g., a blood glucose level). The continuous analyte sensor device 106 may be suitably configured and deployed to monitor any analyte of interest. The non-limiting embodiment described here, however, is realized as a continuous glucose sensor device. Accordingly, an exemplary continuous glucose sensor device is described below.

An embodiment of a continuous glucose sensor device includes a housing that can be attached to the skin of the user by way of an adhesive patch. The housing may be fabricated from a rigid plastic material that can safely and securely hold electrical components of the continuous glucose sensor device. The continuous glucose sensor device includes a thin film sensor element extending from the housing and through the adhesive patch, which may include a hole for the sensor element to pass through. The sensor element includes a relatively thin and elongated element which can be constructed according to so-called thin mask techniques to include elongated conductive elements embedded or encased between layers of a selected insulative sheet material such as a polyimide film or sheet. The proximal end or head of the sensor element is relatively enlarged and defines electrical contacts for electrical coupling to an electronics assembly that resides inside the housing. An opposite or distal segment of the sensor element includes a plurality of exposed sensor electrodes that contact body fluid and/or tissue when the sensor distal segment is placed into the body of the user. The sensor electrodes convey sensor signals representative of the measured analyte level of interest. Thus, the sensor element provides sensor signals that are indicative of the analyte level of the user when the analyte sensor device is deployed at the user.

The sensor signals are transmitted in an ongoing manner from the continuous glucose sensor device (which includes a wireless transmitter) to an appropriate destination device for recordation, processing, and/or display as a monitored patient condition. Referring again to FIG. 1, the continuous analyte sensor device 106 may be suitably configured to transmit sensor signals to the client device 108, the server system 110, and/or to another sensor device, such as the noninvasive sensor device(s) 104. Further description of flexible thin film sensors of this general type may be found in U.S. Pat. No. 5,391,250, which is herein incorporated by reference. Sensor electronics including wireless transmitters are discussed, for example, in U.S. Pat. No. 7,602,310, which is herein incorporated by reference. As mentioned above, the system 100 may support the use of an implantable continuous analyte sensor device that has wireless communication capabilities. Accordingly, an implanted continuous glucose sensor could be employed in addition to, or in lieu of, a sensor device that is designed to be worn on the skin of the patient.

The client device 108 generally represents an electronic device operating within a communication network that supports the system 100. In practice, the client device 108 can be realized as any sort of personal computer, mobile telephone, tablet or other network-enabled electronic device that includes a display device (such as a monitor, screen, or another conventional electronic display that is capable of graphically presenting data and/or information provided by the server system 110), and further includes a user input device, such as a keyboard, a mouse, a touchscreen, or the like, capable of receiving input data and/or other information from the user of the client device 108. In certain embodiments, a user manipulates the client device 108 to execute a client application 116 that contacts the server system 110 via a networking protocol, such as the hypertext transport protocol (HTTP) or the like. The client application 116 may be utilized to access and view data stored in the database system 112, or otherwise receive information or indications pertaining to operation or status of the continuous analyte sensor device 106, the noninvasive sensor device(s) 104, or the like. The client device 108 may include or cooperate with one or more sources of descriptive data 114, which may be implemented as physically distinct devices, or which may be internal to the client device 108. For example, the client device 108 can obtain descriptive data in the form of user input, or it can receive descriptive data from another device operating in the system 100.

The server system 110 is implemented as a computing system or another combination of processing logic, circuitry, hardware, and/or other components configured to support the processes, tasks, operations, and/or functions described herein. In this regard, the server system 110 includes a processing system 120, which may be implemented using any suitable processing system and/or device, such as, for example, one or more processors, central processing units (CPUs), controllers, microprocessors, microcontrollers, processing cores and/or other hardware computing resources configured to support the operation of the processing system 120 described herein. The server system 110 may include or otherwise access a memory (data storage) element 124 having a non-transitory storage medium that is capable of storing programming instructions for execution by the processing system 120. The instructions, when read and executed, cause processing system 120 to perform or otherwise support the processes, tasks, operations, and/or functions described herein. Depending on the embodiment, the memory element 124 may be realized as a random access memory (RAM), read only memory (ROM), flash memory, magnetic or optical mass storage, or any other suitable non-transitory short or long term data storage or other computer-readable media, and/or any suitable combination thereof.

In exemplary embodiments, the server system 110 is coupled to the database system 112, which is configured and arranged to store or otherwise maintain historical measurement data, historical user data, historical control parameter data, and other uploaded operational context or demographic information corresponding to such data in association with the user 102, the client device 108, the continuous analyte sensor device 106, and/or the noninvasive sensor device 104. As described in more detail below, the server system 110 may be deployed as a cloud-based system that performs various tasks related to the calibration and configuration of the noninvasive sensor device 104.

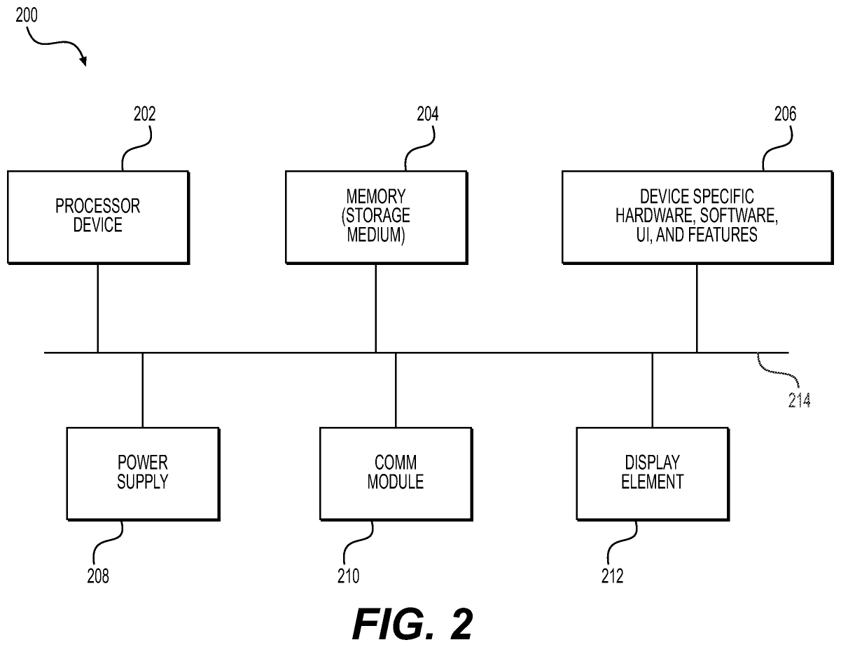

In accordance with certain embodiments, the noninvasive sensor device 104, the continuous analyte sensor device 106, the client device 108, and the server system 110 can be implemented as a computer-based or a processor-based device, system, or component having suitably configured hardware and software written to perform the functions and methods needed to support the features described herein. In this regard, FIG. 2 is a simplified block diagram representation of an exemplary embodiment of a computer-based or processor-based device 200 that is suitable for deployment in the system shown in FIG. 1.

The illustrated embodiment of the device 200 is intended to be a high-level and generic representation of one suitable platform. In this regard, any computer-based or processor-based component of the system 100 can utilize the architecture of the device 200. The illustrated embodiment of the device 200 generally includes, without limitation: at least one processor device 202; a suitable amount of memory 204; device-specific hardware, software, firmware, user interface (UI), and/or features 206; a power supply 208 such as a disposable or rechargeable battery; a communication module 210; and a display element 212. Of course, an implementation of the device 200 may include additional elements, components, modules, and functionality configured to support various features that are unrelated to the subject matter described here. For example, the device 200 may include certain features and elements to support conventional functions that might be related to the particular implementation and deployment of the device 200. In practice, the elements of the device 200 may be coupled together via a bus or any suitable interconnection architecture 214.

The processor device 202 may be implemented or performed with a general purpose processor, a content addressable memory, a digital signal processor, an application specific integrated circuit, a field programmable gate array, any suitable programmable logic device, discrete gate or transistor logic, discrete hardware components, or any combination designed to perform the functions described here. Moreover, the processor device 202 may be implemented as a combination of computing devices, e.g., a combination of a digital signal processor and a microprocessor, a plurality of microprocessors, one or more microprocessors in conjunction with a digital signal processor core, or any other such configuration.

The memory 204 may be realized as RAM memory, flash memory, EPROM memory, EEPROM memory, registers, a hard disk, a removable disk, a CD-ROM, or any other form of storage medium known in the art. In this regard, the memory 204 can be coupled to the processor device 202 such that the processor device 202 can read information from, and write information to, the memory 204. In the alternative, the memory 204 may be integral to the processor device 202. As an example, the processor device 202 and the memory 204 may reside in an ASIC. At least a portion of the memory 204 can be realized as a computer storage medium that is operatively associated with the processor device 202, e.g., a tangible, non-transitory computer-readable medium having computer-executable instructions stored thereon. The computer-executable instructions are configurable to cause the processor device 202 to perform certain tasks, operations, functions, and processes that are specific to the particular embodiment. In this regard, the memory 204 may represent one suitable implementation of such computer-readable media. Alternatively or additionally, the device 200 could receive and cooperate with computer-readable media (not separately shown) that is realized as a portable or mobile component or platform, e.g., a portable hard drive, a USB flash drive, an optical disc, or the like.

The device-specific hardware, software, firmware, UI, and features 206 may vary from one embodiment of the device 200 to another. For example, the device-specific hardware, software, firmware, UI, and features 206 will support: sensor device operations when the device 200 is realized as a sensor device; smartphone features and functionality when the device 200 is realized as a smartphone; etc. In practice, certain portions or aspects of the device-specific hardware, software, firmware, UI, and features 206 may be implemented in one or more of the other blocks depicted in FIG. 2.

If present, the UI of the device 200 (e.g., a device-specific UI 206) may include or cooperate with various features to allow a user to interact with the device 200. Accordingly, the UI may include various human-to-machine interfaces, e.g., a keypad, keys, a keyboard, buttons, switches, knobs, a touchpad, a joystick, a pointing device, a virtual writing tablet, a touch screen, a microphone, or any device, component, or function that enables the user to select options, input information, or otherwise control the operation of the device 200. The UI may include one or more graphical user interface (GUI) control elements that enable a user to manipulate or otherwise interact with an application via the display element 212.

The communication module 210 facilitates data communication between the device 200 and other components as needed during the operation of the device 200. In the context of this description, the communication module 210 can be employed to transmit or stream device-related control data, patient-related data, device-related status or operational data, sensor data, calibration data, and the like. It should be appreciated that the particular configuration and functionality of the communication module 210 can vary depending on the hardware platform and specific implementation of the device 200. Accordingly, with reference to FIG. 1, the communication module of the sensor devices 104, 106 can be utilized to receive and transmit sensor data. Moreover, the communication module of the client device 108 can be used to send and/or receive application data associated with the client application 116. In practice, an embodiment of the device 200 may support wireless data communication and/or wired data communication, using various data communication protocols. For example, the communication module 210 could support one or more wireless data communication protocols, techniques, or methodologies, including, without limitation: RF; IrDA (infrared); Bluetooth; ZigBee (and other variants of the IEEE 802.15 protocol); IEEE 802.11 (any variation); IEEE 802.16 (WiMAX or any other variation); Direct Sequence Spread Spectrum; Frequency Hopping Spread Spectrum; cellular/wireless/cordless telecommunication protocols; wireless home network communication protocols; paging network protocols; magnetic induction; satellite data communication protocols; wireless hospital or health care facility network protocols such as those operating in the WMTS bands; GPRS; and proprietary wireless data communication protocols such as variants of Wireless USB. Moreover, the communication module 210 could support one or more wired/cabled data communication protocols, including, without limitation: Ethernet; powerline; home network communication protocols; USB; IEEE 1394 (Firewire); hospital network communication protocols; and proprietary data communication protocols.

The display element 212 is suitably configured to enable the device 200 to render and display various screens, recommendation messages, notifications, GUIs, GUI control elements, drop down menus, auto-fill fields, text entry fields, message fields, or the like. Of course, the display element 212 may also be utilized for the display of other information during the operation of the device 200, as is well understood. Notably, the specific configuration, operating characteristics, size, resolution, and functionality of the display element 212 can vary depending upon the practical implementation of the device 200.

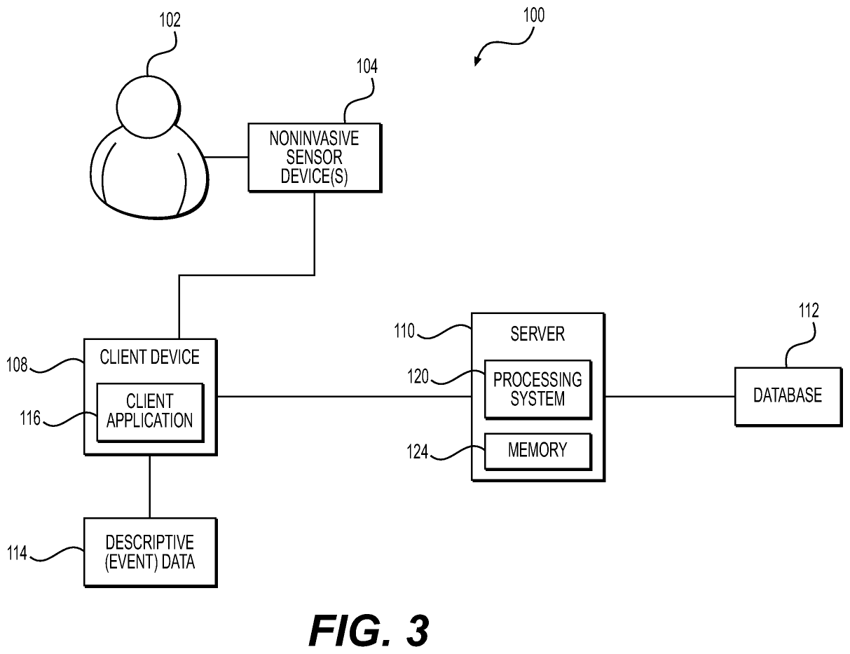

The disclosed subject matter relates to a system and related operating methodology for calibrating, configuring, and operating the noninvasive sensor device 104 in a way that enables it to provide an estimated analyte level of the user 102, in the absence of the continuous analyte sensor device 106. As mentioned above, FIG. 1 depicts the system 100 as deployed during a calibration period of operation. During the calibration period, both of the sensor devices 104, 106 are operated concurrently to obtain respective sensor data. Thereafter, the sensor data is processed (e.g., at the server system 110) to determine a correlation between the analyzed sensor data. If a strong enough correlation is found, a transfer function is generated to obtain estimated analyte levels based on the output of the noninvasive sensor device 104 (without using the continuous analyte sensor device 106). In this regard, FIG. 3 is a block diagram representation of the system 100, as arranged during operation in a measurement mode. The arrangement of the system 100 in FIG. 3 is identical to that shown in FIG. 1, other than the absence of the continuous analyte sensor device 106.

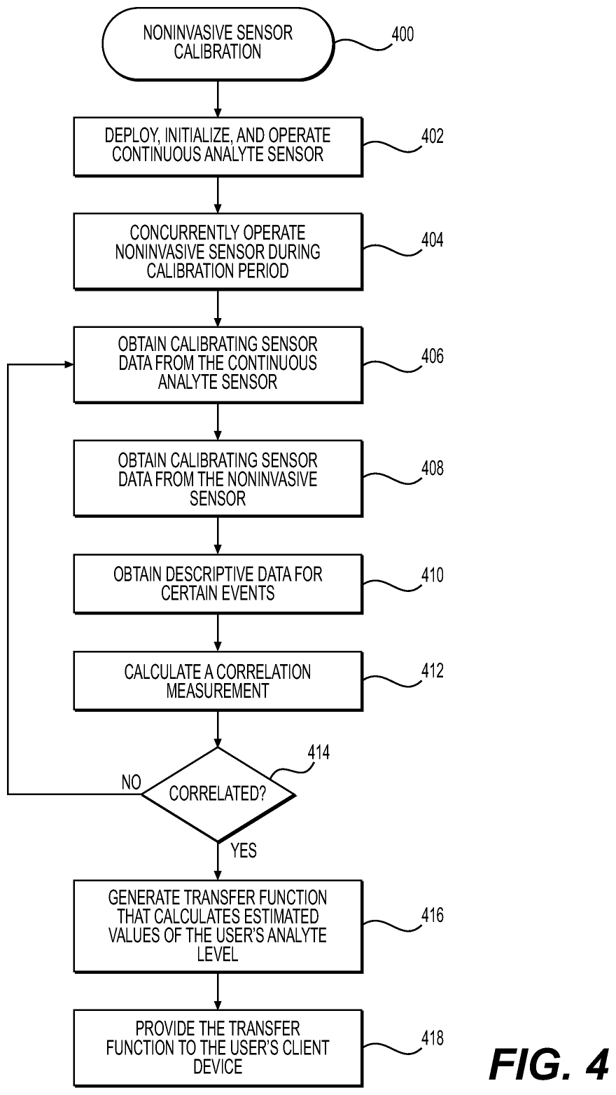

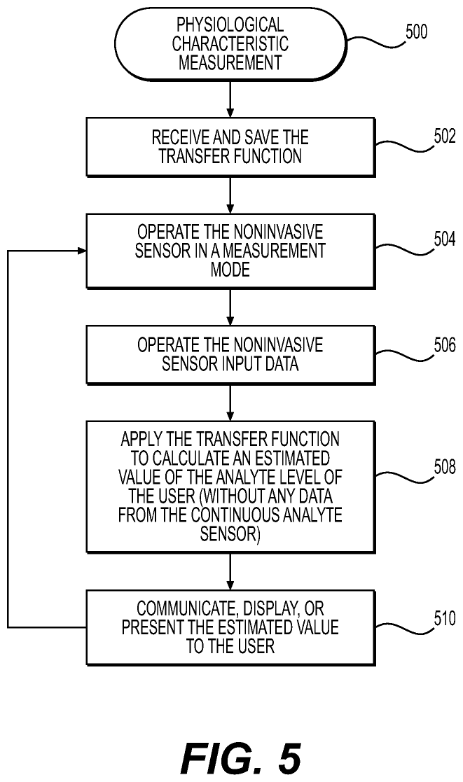

FIG. 4 is a flow chart that illustrates an exemplary embodiment of a process 400 for calibrating a noninvasive physiological characteristic sensor, and FIG. 5 is a flow chart that illustrates an exemplary embodiment of a process 500 for measuring a physiological characteristic of a user (using the calibrated noninvasive physiological characteristic sensor). The various tasks performed in connection with a process described herein may be performed by software, hardware, firmware, or any combination thereof. For illustrative purposes, the description of an illustrated process may refer to elements mentioned above in connection with FIGS. 1-3. It should be appreciated that an illustrated process may include any number of additional or alternative tasks, the tasks shown in a figure need not be performed in the illustrated order, and a process as shown may be incorporated into a more comprehensive procedure or process having additional functionality not described in detail herein. Moreover, one or more of the tasks shown in a figure could be omitted from an embodiment of the disclosed process as long as the intended overall functionality remains intact.

The process 400 can be performed at any appropriate time, and as frequently as necessary. Of course, the process 400 should be performed when a new noninvasive sensor device 104 is activated or first deployed for use. Thereafter, an iteration of the process 400 can be performed on a scheduled basis, on demand by the user (e.g., in response to lifestyle changes, health status changes, a change in weight or diet, or the like), whenever an additional noninvasive sensor device is introduced into the system 100, if the client device 108 determines that recalibration is necessary, if the user 102 repositions or wears the noninvasive sensor device 104 at a different location, etc.

This example assumes that the user already has a compatible and appropriate noninvasive sensor device 104 that is ready for use. Accordingly, the process 400 may begin with the deployment, initialization, and operation of the continuous analyte sensor device 106 (task 402). For example, a continuous glucose sensor device of the type described above can be affixed to the skin of the user 102 and initialized to provide sensor glucose readings/data in an ongoing and continuous manner, in accordance with well-established techniques and operating principles. In certain implementations, the continuous analyte sensor device 106 periodically performs a measurement (e.g., once every five minutes) and sends or stores the corresponding sensor data to a destination device, such as the client device 108.

The noninvasive sensor device 104 and the continuous analyte sensor device 106 are concurrently operated during a calibration period (task 404) to obtain respective measurements, record or store the obtained measurements, and/or transmit the obtained measurements to a destination device, such as the client device 108. The calibration period may be a predefined amount of time, or it may end once the process 400 determines that calibration has been achieved. In practice, however, the calibration period cannot exceed the recommended lifespan of the continuous analyte sensor device 106, which may be several days, a week, ten days, etc. During the calibration period, the sensor devices are operated in a usual manner to obtain respective sensor measurements that are used for calibration purposes. Accordingly, at least task 406, task 408, task 410 (which may be optional), task 412, and query task 414 are performed during the calibration period. For purposes of this example, task 416 and task 418 are also performed during the calibration period, although those tasks may be performed after the calibration period ends. After the calibration period ends, one or both sensor devices may continue operating in a measurement mode, e.g., a period of time following the calibration period, wherein the sensor device(s) operate to generate sensor measurements as usual.

The embodiment of the process 400 described here obtains calibrating sensor data that originates from the continuous analyte sensor device (task 406), wherein the obtained calibrating sensor data is indicative of an analyte level of the user. This calibrating sensor data is produced in response to operation of the continuous analyte sensor device during the calibration period. This calibrating sensor data is indicative of at least one physiological characteristic of the user, such as a blood glucose concentration or level. In practice, this calibrating sensor data includes sensor measurement data corresponding to a plurality of discrete measurement sample times that occur during the calibration period. The obtained calibrating sensor data may be raw sensor signal information, processed measurement information (e.g., information that has been converted, translated, conditioned, filtered, and/or transformed from the raw sensor signal information), or a combination of both raw and processed information.

The process 400 also obtains calibrating sensor data that originates from the noninvasive sensor device (task 408), wherein the obtained calibrating sensor data is indicative of at least one status or condition of the user. This additional calibrating sensor data is produced in response to concurrent operation of the noninvasive sensor device during the common calibration period. This calibrating sensor data can also be indicative of at least one physiological characteristic of the user (which need not be the same physiological characteristic(s) measured by the continuous analyte sensor device). In certain embodiments, the calibrating sensor data produced by the noninvasive sensor device(s) is indicative of a plurality of different measurable conditions, states, or characteristics of the user. In practice, this calibrating sensor data includes sensor measurement data corresponding to a plurality of discrete measurement sample times that occur during the calibration period. The obtained calibrating sensor data may be raw sensor signal information, processed measurement information (e.g., information that has been converted, translated, normalized, conditioned, filtered, and/or transformed from the raw sensor signal information), or a combination of both raw and processed information. Moreover, there may be discrepancies between the sampling rates of the different sensor signals, and pre-processing of the sensor signals can be performed to align or synchronize the sampled sensor data

In a typical implementation, only one continuous analyte sensor device and only one noninvasive sensor device are operated concurrently to generate, collect, and obtain the required sensor data for further analysis and processing. However, one or more additional continuous analyte sensor devices and one or more additional noninvasive sensor device can be utilized to obtain a richer population of calibration data. Alternatively or additionally, the process 400 may obtain any amount of descriptive data for certain events that occur during the calibration period (task 410). Such descriptive data may relate to recordable or measurable characteristics of an event that involves the user, information about the status or condition of the user, operating or environmental conditions or status of the system 100, or the like. As mentioned previously, descriptive data can be user-entered, user-initiated, and/or generated or obtained by a device that operates within the system 100 (such as the client device 108, the server system 110, or any source of descriptive data 114 as depicted in FIG. 1). Usually, such descriptive data is provided by something other than the continuous analyte sensor device 106 or the noninvasive sensor device 104. Descriptive data obtained at task 410 may include any or all of the following, without limitation: information regarding the timing and content of meals; information regarding the mental state or mood of the user; health status of the user, which may include a Ketone value from a blood or urine sample; information regarding the timing and type of exercise performed by the user; GPS data; outside temperature; outside humidity; and calendar or time data.

The system 100 as described here uses calibrating data from the noninvasive sensor device worn by the user. In certain implementations, however, the system 100 can collect and analyze calibrating data from noninvasive sensor devices linked to other users. In other words, the methodologies described here could leverage population data from any number of additional users.

The sensor calibration data (and the descriptive data, if available) is generated or produced by the respective source devices. The raw or processed data may be obtained (tasks 406, 408, 410) at a suitably configured system or device that performs additional processing or analysis of the obtained data. In accordance with the exemplary embodiment described here, the data of interest is initially provided to the client device 108, which sends the data to the server system 110 for further processing. In such an embodiment, the obtaining steps (tasks 406, 408, 410) are performed by the server system 110. In alternative embodiments, the data of interest is obtained by the client device 108, which also performs the additional processing as needed. In accordance with an alternative embodiment, the noninvasive sensor data used for calibration purposes is obtained from a third-party provider, a service, or the like. For example, the manufacturer or distributor of an activity tracker device may permit access to its user database of data collected from its deployed devices.

The process 400 continues by calculating a correlation measurement for the obtained data (task 412). The correlation measurement can be calculated by comparing the calibrating sensor data obtained from the continuous analyte sensor device against the counterpart calibrating sensor data obtained from the noninvasive sensor device. If the collection and processing of descriptive event data is supported, then task 412 can also consider the obtained descriptive data when calculating the correlation measurement. In accordance with well known methodologies and principles, the correlation measurement calculated at task 412 will be a number between 0.0 (indicating no correlation) and 1.0 (indicating complete or full correlation). This description assumes that the server system 110 utilizes machine learning to calculate the correlation measurement. It should be noted that any number of different machine learning techniques may be utilized by the process 400 to calculate the correlation measurement, such as, for example, artificial neural networks, genetic programming, support vector machines, Bayesian networks, probabilistic machine learning models, or other Bayesian techniques, fuzzy logic, heuristically derived combinations, or the like.

Artificial intelligence is an area of computer science emphasizes the creation of intelligent machines that work and react like humans. Some of the activities computers with artificial intelligence are designed for include learning. Examples of artificial intelligence algorithms include, but are not limited to, key learning, actor critic methods, reinforce, deep deterministic policy gradient (DDPG), multi-agent deep deterministic policy gradient (MADDPG), etc. Machine learning refers to an artificial intelligence discipline geared toward the technological development of human knowledge.

Machine learning facilitates a continuous advancement of computing through exposure to new scenarios, testing and adaptation, while employing pattern and trend detection for improved decisions and subsequent, though not identical, situations. Machine learning (ML) algorithms and statistical models can be used by computer systems to effectively perform a specific task without using explicit instructions, relying on patterns and inference instead. Machine learning algorithms build a mathematical model based on sample data, known as “training data,” in order to make predictions or decisions without being explicitly programmed to perform the task. Machine learning algorithms can be used when it is infeasible to develop an algorithm of specific instructions for performing the task.

For example, supervised learning algorithms build a mathematical model of a set of data that contains both the inputs and the desired outputs. The data is known as training data and consists of a set of training examples. Each training example has one or more inputs and a desired output, also known as a supervisory signal. In the case of semi-supervised learning algorithms, some of the training examples are missing the desired output. In the mathematical model, each training example is represented by an array or vector, and the training data by a matrix. Through iterative optimization of an objective function, supervised learning algorithms learn a function that can be used to predict the output associated with new inputs. An optimal function will allow the algorithm to correctly determine the output for inputs that were not a part of the training data. An algorithm that improves the accuracy of its outputs or predictions over time is said to have learned to perform that task. Supervised learning algorithms include classification and regression. Classification algorithms are used when the outputs are restricted to a limited set of values, and regression algorithms are used when the outputs may have any numerical value within a range. Similarity learning is an area of supervised machine learning closely related to regression and classification, but the goal is to learn from examples using a similarity function that measures how similar or related two objects are.

Reinforcement learning is an area of machine learning concerned with how software agents ought to take actions in an environment so as to maximize some notion of cumulative reward. Due to its generality, the field is studied in many other disciplines, such as game theory, control theory, operations research, information theory, simulation-based optimization, multi-agent systems, swarm intelligence, statistics and genetic algorithms. In machine learning, the environment is typically represented as a Markov Decision Process (MDP). Many reinforcement learning algorithms use dynamic programming techniques. Reinforcement learning algorithms do not assume knowledge of an exact mathematical model of the MDP and are used when exact models are infeasible.

In predictive modeling and other types of data analytics, a single model based on one data sample can have biases, high variability or outright inaccuracies that can affect the reliability of its analytical findings. By combining different models or analyzing multiple samples, the effects of those limitations can be reduced to provide better information. As such, ensemble methods can use multiple machine learning algorithms to obtain better predictive performance than could be obtained from any of the constituent learning algorithms alone.

An ensemble is a supervised learning algorithm because it can be trained and then used to make predictions. The trained ensemble, therefore, represents a single hypothesis that is not necessarily contained within the hypothesis space of the models from which it is built. Thus, ensembles can be shown to have more flexibility in the functions they can represent. An ensemble model can include a set of individually trained classifiers (such as neural networks or decision trees) whose predictions are combined.

For instance, one common example of ensemble modeling is a random forest model which is a type of analytical model that leverages multiple decision trees and is designed to predict outcomes based on different variables and rules. A random forest model blends decision trees that may analyze different sample data, evaluate different factors or weight common variables differently. The results of the various decision trees are then either converted into a simple average or aggregated through further weighting. The emergence of Hadoop and other big data technologies has allowed greater volumes of data to be stored and analyzed, which can allow analytical models to be run on different data samples.

Depending on the implementation, any number of machine learning models can be combined to optimize the ensemble model. Examples of machine learning algorithms or models that can be implemented at the machine learning model can include, but are not limited to: regression models such as linear regression, logistic regression, and K-means clustering; one or more decision tree models (e.g., a random forest model); one or more support vector machines; one or more artificial neural networks; one or more deep learning networks (e.g., at least one recurrent neural network, sequence to sequence mapping using deep learning, sequence encoding using deep learning, etc.); fuzzy logic based models; genetic programming models; Bayesian networks or other Bayesian techniques, probabilistic machine learning models; Gaussian processing models; Hidden Markov models; time series methods such as Autoregressive Moving Average (ARMA) models, Autoregressive Integrated Moving Average (ARIMA) models, Autoregressive conditional heteroskedasticity (ARCH) models; generalized autoregressive conditional heteroskedasticity (GARCH) models; moving-average (MA) models or other models; and heuristically derived combinations of any of the above, etc. The types of machine learning algorithms differ in their approach, the type of data they input and output, and the type of task or problem that they are intended to solve.

A Hidden Markov model (HMM) is a statistical Markov model in which the system being modeled is assumed to be a Markov process with unobserved (hidden) states. An HMM can be considered as the simplest dynamic Bayesian network. A Bayesian network, belief network or directed acyclic graphical model is a probabilistic graphical model that represents a set of random variables and their conditional independence with a directed acyclic graph (DAG). Bayesian networks that model sequences of variables are called dynamic Bayesian networks. Generalizations of Bayesian networks that can represent and solve decision problems under uncertainty are called influence diagrams.

Support vector machines (SVMs), also known as support vector networks, are a set of related supervised learning methods used for classification and regression. Given a set of training examples, each marked as belonging to one of two categories, an SVM training algorithm builds a model that predicts whether a new example falls into one category or the other. An SVM training algorithm is a non-probabilistic, binary, linear classifier. In addition to performing linear classification, SVMs can efficiently perform a non-linear classification using what is called the kernel trick, implicitly mapping their inputs into high-dimensional feature spaces.

Decision tree learning uses a decision tree as a predictive model to go from observations about an item (represented in the branches) to conclusions about the item's target value (represented in the leaves). Tree models where the target variable can take a discrete set of values are called classification trees; in these tree structures, leaves represent class labels and branches represent conjunctions of features that lead to those class labels. Decision trees where the target variable can take continuous values (typically real numbers) are called regression trees. In decision analysis, a decision tree can be used to visually and explicitly represent decisions and decision making.

Deep learning algorithms can refer to a collection of algorithms used in machine learning, that are used to model high-level abstractions and data through the use of model architectures, which are composed of multiple nonlinear transformations. Deep learning is a specific approach used for building and training neural networks. Deep learning consists of multiple hidden layers in an artificial neural network. Examples of deep learning algorithms can include, for example, Siamese networks, transfer learning, recurrent neural networks (RNNs), long short-term memory (LSTM) networks, convolutional neural networks (CNNs), transformers, etc. For instance, deep learning approaches can make use of autoregressive Recurrent Neural Networks (RNN), such as the long short-term memory (LSTM) and the Gated Recurrent Unit (GRU). One neural network architecture for time series forecasting using RNNs (and variants) is an autoregressive seq2seq neural network architecture, which acts as an autoencoder.

In some embodiments, the ensemble model can include one or more deep learning algorithms. It should be noted that any number of different machine learning techniques may also be utilized. Depending on the implementation, the ensemble model can be implemented as a bootstrap aggregating ensemble algorithm (also referred to as a bagging classifier method), as a boosting ensemble algorithm or classifier algorithm, as a stacking ensemble algorithm or classifier algorithm, as bucket of models ensemble algorithms, as Bayes optimal classifier algorithms, as Bayesian parameter averaging algorithms, as Bayesian model combination algorithms, etc.

Bootstrap aggregating, often abbreviated as bagging, involves having each model in the ensemble vote with equal weight. In order to promote model variance, bagging trains each model in the ensemble using a randomly drawn subset of the training set. As an example, the random forest algorithm combines random decision trees with bagging to achieve very high classification accuracy. A bagging classifier or ensemble method creates individuals for its ensemble by training each classifier on a random redistribution of the training set. Each classifier's training set can be generated by randomly drawing, with replacement, N examples—where N is the size of the original training set; many of the original examples may be repeated in the resulting training set while others may be left out. Each individual classifier in the ensemble is generated with a different random sampling of the training set. Bagging is effective on “unstable” learning algorithms (e.g., neural networks and decision trees), where small changes in the training set result in large changes in predictions.

By contrast, boosting involves incrementally building an ensemble by training each new model instance to emphasize the training instances that previous models mis-classified. In some cases, boosting has been shown to yield better accuracy than bagging, but it also tends to be more likely to over-fit the training data. A boosting classifier can refer to a family of methods that can be used to produce a series of classifiers. The training set used for each member of the series is chosen based on the performance of the earlier classifier(s) in the series. In boosting, examples that are incorrectly predicted by previous classifiers in the series are chosen more often than examples that were correctly predicted. Thus, boosting attempts to produce new classifiers that are better able to predict examples for which the current ensemble's performance is poor. A common implementation of boosting is Adaboost, although some newer algorithms are reported to achieve better results.

Stacking (sometimes called stacked generalization) involves training a learning algorithm to combine the predictions of several other learning algorithms. Stacking works in two phases: multiple base classifiers are used to predict the class, and then a new learner is used to combine their predictions with the aim of reducing the generalization error. First, all of the other algorithms are trained using the available data, then a combiner algorithm is trained to make a final prediction using all the predictions of the other algorithms as additional inputs. If an arbitrary combiner algorithm is used, then stacking can theoretically represent any of the ensemble techniques described in this article, although, in practice, a logistic regression model is often used as the combiner.

A “bucket of models” is an ensemble technique in which a model selection algorithm is used to choose the best model for each problem. When tested with only one problem, a bucket of models can produce no better results than the best model in the set, but when evaluated across many problems, it will typically produce much better results, on average, than any model in the set. One common approach used for model-selection is cross-validation selection (sometimes called a “bake-off contest”). Cross-validation selection can be summed up as try them all with the training set and pick the one that works best. Gating is a generalization of Cross-Validation Selection. It involves training another learning model to decide which of the models in the bucket is best-suited to solve the problem. Often, a perceptron is used for the gating model. It can be used to pick the “best” model, or it can be used to give a linear weight to the predictions from each model in the bucket. When a bucket of models is used with a large set of problems, it may be desirable to avoid training some of the models that take a long time to train. Landmark learning is a meta-learning approach that seeks to solve this problem. It involves training only the fast (but imprecise) algorithms in the bucket, and then using the performance of these algorithms to help determine which slow (but accurate) algorithm is most likely to do best.

The Bayes optimal classifier is a classification technique. It is an ensemble of all the hypotheses in the hypothesis space. On average, no other ensemble can outperform it. The naive Bayes optimal classifier is a version of this that assumes that the data is conditionally independent on the class and makes the computation more feasible. Each hypothesis is given a vote proportional to the likelihood that the training dataset would be sampled from a system if that hypothesis were true. To facilitate training data of finite size, the vote of each hypothesis is also multiplied by the prior probability of that hypothesis. The hypothesis represented by the Bayes optimal classifier, however, is the optimal hypothesis in ensemble space (the space of all possible ensembles.

Bayesian parameter averaging (BPA) is an ensemble technique that seeks to approximate the Bayes optimal classifier by sampling hypotheses from the hypothesis space and combining them using Bayes' law. Unlike the Bayes optimal classifier, Bayesian model averaging (BMA) can be practically implemented. Hypotheses are typically sampled using a Monte Carlo sampling technique such as MCMC. For example, Gibbs sampling may be used to draw hypotheses that are representative of a distribution. It has been shown that under certain circumstances, when hypotheses are drawn in this manner and averaged according to Bayes' law, this technique has an expected error that is bounded to be at most twice the expected error of the Bayes optimal classifier.

Bayesian model combination (BMC) is an algorithmic correction to Bayesian model averaging (BMA). Instead of sampling each model in the ensemble individually, it samples from the space of possible ensembles (with model weightings drawn randomly from a Dirichlet distribution having uniform parameters). This modification overcomes the tendency of BMA to converge toward giving all of the weight to a single model. Although BMC is somewhat more computationally expensive than BMA, it tends to yield dramatically better results. The results from BMC have been shown to be better on average (with statistical significance) than BMA, and bagging.

The use of Bayes' law to compute model weights necessitates computing the probability of the data given each model. Typically, none of the models in the ensemble are exactly the distribution from which the training data were generated, so all of them correctly receive a value close to zero for this term. This would work well if the ensemble were big enough to sample the entire model-space, but such is rarely possible. Consequently, each pattern in the training data will cause the ensemble weight to shift toward the model in the ensemble that is closest to the distribution of the training data. It essentially reduces to an unnecessarily complex method for doing model selection. The possible weightings for an ensemble can be visualized as lying on a simplex. At each vertex of the simplex, all of the weight is given to a single model in the ensemble. BMA converges toward the vertex that is closest to the distribution of the training data. By contrast, BMC converges toward the point where this distribution projects onto the simplex. In other words, instead of selecting the one model that is closest to the generating distribution, it seeks the combination of models that is closest to the generating distribution. The results from BMA can often be approximated by using cross-validation to select the best model from a bucket of models. Likewise, the results from BMC may be approximated by using cross-validation to select the best ensemble combination from a random sampling of possible weightings.

The correlation measurement calculated at task 412 indicates how well the user's analyte level can be estimated using the other obtained data (data from the noninvasive sensor device(s), possibly combined with an amount of descriptive data for certain events). For example, a fitness monitor or activity tracker may provide data that indicates the user's blood density, blood pressure, heart rate, and body temperature (four different types of sensor data) and a mobile device may provide GPS data (a type of descriptive event data), for a total of five different inputs to be correlated to the continuous analyte sensor data (e.g., a blood glucose concentration). Notably, the noninvasive sensor data need not include any readings or direct measurements of the analyte level of interest. Instead, the system 100 is capable of estimating the analyte level based on information obtained from other sensor types and/or other sources of data.

Referring again to FIG. 4, the process 400 checks whether the correlation measurement exceeds a threshold value that indicates a good correlation (query task 414). The threshold value can be selected to suit the needs of the particular embodiment and/or application of the system 100. In accordance with the exemplary deployment described here, the threshold value is set to be 0.8, although other threshold values can be utilized if so desired. If the process 400 determines that the correlation measurement exceeds the threshold value (the “Yes” branch of query task 414), then the process 400 continues by generating a transfer function that calculates estimated values of the analyte level of the user as a function of noninvasive sensor data produced in response to operation of the noninvasive sensor device(s) in a normal measurement mode (task 416). If the calculated correlation measurement is too low (the “No” branch of query task 414), then the process 400 may exit or return to task 406 to continue the calibration routine.

For the system 100 described here, the server system 110 generates the transfer function, which is expressed as an equation for calculating estimated values of the analyte level of interest based on input data or variables that correspond to the different data types considered during the correlation analysis. If a suitable transfer function cannot be generated, then the system 100 can request more data and/or notify the user in an appropriate manner. If the sensor device 106 is at or near the end of life, then a new sensor would need to be deployed, but the study could continue and not exit. If other data, such as descriptive event data, was used in the correlation analysis, then the transfer function will include the other data types or data sources for purposes of calculating the estimated values of the analyte level. In practice, the transfer function can be a combination of linear and non-linear models (depending on the implementation), which are then fused back together such that future input signals or data can be converted into predicted or estimated values of the analyte level. In this way, the noninvasive sensor device 104 can serve as a surrogate for the continuous analyte sensor device 106.

This description assumes that the server system 110 generates a suitable transfer function at task 416, and provides the transfer function to the user's client device 108 (task 418). The client device 108 receives and saves the transfer function for use with the client application 116 going forward. In alternative embodiments, the server system 110 maintains and applies the transfer function as needed to calculate the estimated values of the analyte level. In such embodiments, the server system 110 communicates the estimated values of the user's analyte level to the client device 108 and/or to another device or system available to the user.

The following description assumes that the client device 108 performs the measurement process 500. Accordingly, the process 500 begins with the client device 108 receiving and saving the transfer function (task 502), as mentioned above. The noninvasive sensor device 104, which was also used during the calibration process 400, is operated in a conventional measurement mode (task 504) to obtain corresponding noninvasive sensor input data (task 506). As explained above, the measurement mode is different than the calibration mode in that sensor measurements obtained during the measurement mode need not be used for purposes of calibration and are instead intended for use in monitoring the analyte level, assisting with therapy dosages, and the like. The noninvasive sensor input data is produced in response to operation of the noninvasive sensor device(s) 104 in the measurement mode, and is indicative of at least one physiological characteristic of the user, one or more measurable conditions or states of the user, or the like. This example assumes that descriptive event data was not utilized during the calibration process 400 and, therefore, that the saved transfer function does not contemplate or accommodate descriptive data as an input. If, however, the transfer function requires descriptive data as an input, then the process 500 will also obtain corresponding descriptive input data while the noninvasive sensor device 104 is operating in the measurement mode.

Next, the client device 108 uses and applies the saved transfer function to calculate an estimated value of the analyte level of the user (task 508). The estimated value is calculated from the obtained noninvasive sensor input data. More specifically, the noninvasive sensor input data serves as the input to the transfer function, and the output of the transfer function is the estimated value of the analyte level. For this particular embodiment, the estimated value is calculated in the absence of any data from the continuous analyte sensor device. Although not required, this example assumes that the continuous analyte sensor device was removed or disabled following completion of the calibration process 400.

The process 510 continues by communicating, displaying, or otherwise presenting the estimated value to the user (task 510). For this particular embodiment, the client device 108 communicates the estimated value by way of a suitably formatted display screen, notification, message, or alert. As shown in FIG. 5, tasks 504, 506, 508, and 510 can be repeated as needed to obtain updated estimated values of the analyte level. Moreover, the client device 108 can be operated to generate a plot, table, or chart of the estimated values over time.

The system 100 and related calibration and measurement methodologies can be effectively utilized to increase the accuracy of noninvasive analyte monitoring, such as glucose monitoring, by calibrating the noninvasive sensor device and measurements with continuous analyte sensor values that were collected during a calibration period. After calibration is completed, the user can continue to monitor the analyte levels using only the noninvasive sensor device that was used during the calibration phase.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application.

What is claimed is:

1. A method of measuring a physiological characteristic of a user, the method comprising the steps of:
obtaining first calibrating sensor data that is indicative of an analyte level of the user, the first calibrating sensor data produced in response to operation of a continuous analyte sensor device during a calibration period;
obtaining second calibrating sensor data that is indicative of status or condition of the user, the second calibrating sensor data produced in response to operation of at least one noninvasive sensor device, concurrently with operation of the continuous analyte sensor device, during the calibration period;
calculating a correlation measurement by comparing the obtained first calibrating sensor data against the obtained second calibrating sensor data;
when the correlation measurement exceeds a threshold value, generating a transfer function that calculates estimated values of the analyte level of the user as a function of noninvasive sensor data produced in response to operation of the at least one noninvasive sensor device;
after generating the transfer function, obtaining noninvasive sensor input data that is indicative of status or condition of the user, the noninvasive sensor input data produced in response to operation of the at least one noninvasive sensor device in a measurement mode;
applying the transfer function to calculate, from the obtained noninvasive sensor input data, an estimated value of the analyte level of the user; and
communicating the estimated value of the analyte level for presentation to the user.

2. The method of claim 1, wherein:
the second calibrating sensor data is indicative of at least one physiological characteristic of the user; and
the noninvasive sensor input data is indicative of the at least one physiological characteristic of the user.

3. The method of claim 1, wherein:
the second calibrating sensor data is indicative of a plurality of different measurable conditions of the user; and
the noninvasive sensor input data is indicative of the plurality of different measurable conditions of the user.

4. The method of claim 1, wherein:
the continuous analyte sensor device comprises a continuous glucose sensor device; and
the first calibrating sensor data is indicative of a blood glucose level of the user.

5. The method of claim 1, wherein the at least one noninvasive sensor device comprises a wellness monitoring device, a health monitoring device, a physiological property monitoring device, or an activity tracker device.

6. The method of claim 1, further comprising the step of providing the transfer function to a client device associated with the user, wherein:
the step of obtaining noninvasive sensor input data is performed by the noninvasive sensor device or the client device; and
the step of applying the transfer function is performed by the client device.

7. The method of claim 6, wherein the step of communicating the estimated value of the analyte level is performed by the client device.

8. The method of claim 1, further comprising the step of obtaining descriptive data for events that occur during the calibration period, wherein:
the calculating step calculates the correlation measurement by comparing the obtained first calibrating sensor data against the obtained second calibrating sensor data and the obtained descriptive data.

9. The method of claim 1, wherein the step of applying the transfer function calculates the estimated value of the analyte in the absence of any data from the continuous analyte sensor device.

10. A non-transitory computer-readable storage medium comprising program instructions stored thereon, wherein the program instructions are configurable to cause at least one processor device to perform a method comprising the steps of:
obtaining first calibrating sensor data that is indicative of an analyte level of the user, the first calibrating sensor data produced in response to operation of a continuous analyte sensor device during a calibration period;
obtaining second calibrating sensor data that is indicative of status or condition of the user, the second calibrating sensor data produced in response to operation of at least one noninvasive sensor device, concurrently with operation of the continuous analyte sensor device, during the calibration period;
calculating a correlation measurement by comparing the obtained first calibrating sensor data against the obtained second calibrating sensor data;
when the correlation measurement exceeds a threshold value, generating a transfer function that calculates estimated values of the analyte level of the user as a function of noninvasive sensor data produced in response to operation of the at least one noninvasive sensor device; and
providing the generated transfer function to a client device associated with the user.

11. The storage medium of claim 10, wherein:
the client device uses the generated transfer function to calculate, from obtained noninvasive sensor input data that is indicative of status or condition of the user, an estimated value of the analyte level of the user;
the noninvasive sensor input data is produced in response to operation of the at least one noninvasive sensor device in a measurement mode; and
the client device communicates the estimated value of the analyte level to the user.

12. The storage medium of claim 10, wherein the second calibrating sensor data is indicative of a plurality of different measurable conditions of the user.

13. The storage medium of claim 10, wherein:
the continuous analyte sensor device comprises a continuous glucose sensor device; and
the first calibrating sensor data is indicative of a blood glucose level of the user.

14. The storage medium of claim 10, wherein the at least one noninvasive sensor device comprises a wellness monitoring device, a health monitoring device, a physiological property monitoring device, or an activity tracker device.

15. A system to measure a physiological characteristic of a user, the system comprising:
at least one processor device; and
a non-transitory processor-readable medium operatively associated with the at least one processor device, the processor-readable medium comprising executable program instructions configurable to cause the at least one processor device to perform a method comprising the steps of:
obtaining first calibrating sensor data that is indicative of an analyte level of the user, the first calibrating sensor data produced in response to operation of a continuous analyte sensor device during a calibration period;
obtaining second calibrating sensor data that is indicative of status or condition of the user, the second calibrating sensor data produced in response to operation of at least one noninvasive sensor device, concurrently with operation of the continuous analyte sensor device, during the calibration period;
calculating a correlation measurement by comparing the obtained first calibrating sensor data against the obtained second calibrating sensor data;
when the correlation measurement exceeds a threshold value, generating a transfer function that calculates estimated values of the analyte level of the user as a function of noninvasive sensor data produced in response to operation of the at least one noninvasive sensor device; and
providing the generated transfer function to a client device associated with the user;
wherein the client device obtains noninvasive sensor input data that is indicative of status or condition of the user, the noninvasive sensor input data produced in response to operation of the at least one noninvasive sensor device in a measurement mode; and
wherein the client device applies the transfer function to calculate, from the obtained noninvasive sensor input data, an estimated value of the analyte level of the user.

16. The system of claim 15, wherein the client device communicates the estimated value of the analyte level to the user.

17. The system of claim 15, wherein the second calibrating sensor data is indicative of a plurality of different measurable conditions of the user.

18. The system of claim 15, wherein:
the continuous analyte sensor device comprises a continuous glucose sensor device; and
the first calibrating sensor data is indicative of a blood glucose level of the user.

19. The system of claim 15, wherein the at least one noninvasive sensor device comprises a wellness monitoring device, a health monitoring device, or an activity tracker device.

20. The system of claim 15, wherein the client device applies the transfer function to calculate the estimated value of the analyte in the absence of any data from the continuous analyte sensor device.
